(19) United States
(12) Patent Application Publication
(10) Pub. No.: US 2025/0375510 A1
(43) Pub. Date: Dec. 11, 2025

(54) VACCINE FOR PROTECTION AGAINST LEPTOSPIRA SEROVAR GRIPPOTYPHOSA

(71) Applicant: INTERVET INC., Rahway, NJ (US)

(72) Inventors: Henricus Leo Bernardus Maria KLAASEN, Wijchen (NL); Jorrit HEGGE, Arnhem (NL)

(73) Assignee: INTERVET INC., Rahway, NJ (US)

(21) Appl. No.: 18/873,754

(22) PCT Filed: Jun. 21, 2023
(86) PCT No.: PCT/EP2023/066785

§ 371 Date: Dec. 11, 2024

(30) Foreign Application Priority Data
Jun. 22, 2022 (EP) 22180306.7

(51) Int. Cl. A61K 39/02; A61K 39/00

(52) U.S. Cl. CPC A61K 39/0225; A61K 2039/545

(57) ABSTRACT

The present invention is directed to providing a canine with protective immunity against Leptospira serovar Grippoty-phosa with a vaccine that comprises a non-Grippotyphosa Leptospira serovar.

### FIELD OF THE INVENTION

The present invention relates generally to immunogenic Leptospira compositions, which are capable of eliciting cross-protective immune responses in animals, particularly canine animals. The invention further relates to methods of providing animals, especially canine animals, with cross-protective immune responses against Leptospira Grippotyphosa.

### BACKGROUND OF THE INVENTION

Leptospirosis is a worldwide zoonotic disease caused by gram-negative spirochetes belonging to the genus Leptospira. Leptospirosis is prevalent in humans, dogs, horses, cattle and wild animals. Dogs are highly susceptible to infection and become ill after infection with symptoms such as high fever, jaundice, hemorrhagic diathesis, abortion and can die within days. In addition, the dog may develop chronic symptoms such as liver, kidney and gastrointestinal symptoms. Domestic dogs live in close association with people and livestock and can be used as a sentinel species for the environmental risk to humans.

Seroprevalence studies suggest that the predominant and most widespread serogroups in dogs are Canicola, Icterohaemorrhagiae, Australis, and Grippotyphosa, with, in addition to these serogroups, Pomona being relevant in the USA, and Hebdomadis in Japan. A range of vaccines against canine leptospirosis have been licensed in Europe, the oldest vaccines being bivalent vaccines containing serovars Canicola and either Icterohaemorrhagiae or Copenhageni. More recently, tetravalent and trivalent vaccines were introduced onto the European market, in which also serovars of serogroups Grippotyphosa and Australis or Grippotyphosa alone were included.

Review of the Literature, for example “Leptospirosis Fact Sheet” (WHO, Regional Office for South-East Asia, 2009) indicates, in part, that animals and humans can be immunized, but that protection is largely serovar-specific. Lack of cross-protection is not surprising, particularly in view of the significant genetic/genomic differences between the serovars, for example, among the gene organization in the lipopolysaccharide biosynthetic (rfb) locus (Pena-Moctezuma, A. et al, 2001 FEMS Immunology and Medical Microbiology 31 (2001) 73-81).

There is thus little if any evidence for “cross-protection”, between serovars in canines. Cross-protection or heterologous protection is herein defined as providing protection against a Leptospira serovar by administering an effective amount of a different serovar (e.g. protecting against Grippotyphosa serovar by administering a homologous effective amount of a non-Grippotyphosa serovar, e.g. serovar Portland-vere, Dadas, Copenhageni, or Bratislava).

EP2874653 discloses methods for providing protection against Leptospira interrogans serovar Copenhageni using a multivalent vaccine comprising non-Copenhageni serovars Icterohaemorrhagiae, Canicola, Grippotyphosa, and Pomona.

This was the first ever disclosure of a Leptospira vaccine that provided protection in dogs against a serovar that was not present in the vaccine. This patent was published in January 2014 and since then no other cross-protection of Leptospira serovars has been reported in dogs.

An article by Bouvet et al. (Veterinary Immunology and Immunopathology 219 (2020) 109985) is the scientific publication of the same findings and experiments as described in EP2874653. In there, it is confirmed that bacterin Leptospira vaccines only provide protection against the same serovar. Also it is disclosed that there is very limited proof available of protection against other serovars within the same serogroup and only in rodent models. Proof of efficacy in dogs of other Leptospira serovars providing cross-protection has not been shown in the prior art.

There is thus still a need to protect animals, in particular dogs, against Leptospira serovars. With Leptospirosis being also a potential zoonotic problem for humans, it would be beneficial to have protection against additional Leptospira serovars, and preferably with the vaccines already used. It would therefore be beneficial to protect animals, in particular dogs, against heterologous Leptospira serovars Until the present disclosure, methods for providing protection against Leptospira serovar Grippotyphosa using non-Grippotyphosa serovars were not known.

### SUMMARY OF THE INVENTION

An object of this invention is to provide methods for providing protective immunity against a first Leptospira serovar comprising the step of administering a further Leptospira serovar(s), which is a different serovar, with respect to the first Leptospira serovar. In the cases where the further Leptospira serovar(s) is a combination of Leptospira serovars (e.g. a combination/multi-valent vaccine), the further Leptospira serovar(s) must not contain a Leptospira serovar of the same serovar as the first Leptospira serovar, for which protective immunity is being sought.

In an embodiment of the invention and/or embodiments thereof, the methods provide protective immunity against Leptospira serovar Grippotyphosa, and comprise the step of administering an immunologically effective amount of a non-Grippotyphosa Leptospira serovar to an animal in need thereof.

In another embodiment, the methods provide protective immunity against Leptospira serovar Grippotyphosa by administering a combination/multivalent Leptospira vaccine. In a particular embodiment, the multivalent Leptospira vaccine comprises Leptospira serovars Portland-vere, Dadas, Copenhageni, and Bratislava. Nobivac L4 (MSD Animal Health) is such a multivalent vaccine.

It was unexpected and surprising to the skilled worker in possession of the current state-of-the-art knowledge in the field of leptospirosis, that a vaccine that does not contain Leptospira serovar Grippotyphosa elicits protective immunity against Leptospira serovar Grippotyphosa in canines. In particular it was surprising that a vaccine comprising Leptospira serovars Portland-vere, Dadas, Copenhageni, and Bratislava provided immunity against Leptospira serovar Grippotyphosa.

The examples show that a vaccine comprising Leptospira serovars Portland-vere, Dadas, Copenhageni, and Bratislava such as Nobivac L4, provided protective immunity against Leptospira serovar Grippotyphosa.

### DETAILED DESCRIPTION OF THE INVENTION

The present invention encompasses methods for prevention or reduction of infection due to Leptospira of a particular serovar by administering a vaccine with one or more Leptospira of a different serovar. The present invention is directed to a vaccine composition comprising a Leptospira serovar for use in providing a canine with protective immunity against Leptospira serovar Grippotyphosa. Said vaccine does not comprise the Leptospira serovar Grippotyphosa.

In an embodiment of the invention and/or embodiments thereof, the invention provides methods of eliciting in an animal a protective immune response against Leptospira serovar Grippotyphosa comprising the step of administering to the animal an effective amount of a non-Grippotyphosa Leptospira serovar. “Non-Grippotyphosa Leptospira serovar” means a Leptospira serovar that is different from serovar Grippotyphosa. It can be from the same or different serogroup as serovar Grippotyphosa (serogroup Grippotyphosa).

In an embodiment of the invention and/or embodiments thereof, the non-Grippotyphosa Leptospira serovar belongs to the serogroup Grippotyphosa. In a particular embodiment, the non-Grippotyphosa Leptospira serovar is Dadas or Bananal/Liangguang, preferably Dadas.

In an embodiment of the invention and/or embodiments thereof, the non-Grippotyphosa Leptospira serovar is delivered as part of a multivalent/combination vaccine. In a particular embodiment, the non-Grippotyphosa Leptospira serovar is a serovar selected from the group consisting of Leptospira Portland-vere, Dadas, Copenhageni, and Bratislava, preferably serovar Dadas and one or more serovars selected from Portland-vere, Copenhageni and Bratislava.

In another embodiment of the invention and/or embodiments thereof, the vaccine comprises Leptospira serovar Portland-vere. In another embodiment, the vaccine comprises Leptospira serovar Dadas. In another embodiment, the vaccine comprises Leptospira serovar Copenhageni. In another embodiment, the vaccine comprises Leptospira serovar Bratislava. In still another embodiment, the vaccine comprises Leptospira serovars Portland-vere, Dadas, Copenhageni, and Bratislava.

Suitably, Leptospira Portland-vere strain is Ca-12-000. Suitably the Leptospira Dadas strain is Gr-01-005. Suitably the Leptospira Copenhageni strain is Ic-02-001. Suitably the Leptospira Bratislava strain is As-05-073.

Suitably, the vaccine provides a canine with protective immunity against Leptospira serovar Grippotyphosa, Australis, and Icterohaemorrhagiae.

The term vaccine as used herein refers to a pharmaceutical composition comprising at least one immunologically active component that induces an immunological response in an animal and a pharmaceutically acceptable carrier. A vaccine may also be referred to as an immunogenic composition in the present specification. A vaccine may additionally comprise further components typical to pharmaceutical compositions. An immunogenic composition and a vaccine is used interchangeably in the present specification.

Usually, an “immunological response” includes but is not limited to one or more of the following effects: the production or activation of antibodies, B cells, helper T cells, suppressor T cells, and/or cytotoxic T cells and/or gamma-delta T cells, directed specifically to an antigen or antigens included in the composition or vaccine of interest. Suitably, the target will display either a therapeutic or preventive immunological response such that resistance to new infection will be enhanced and/or the clinical severity of the disease is reduced. Such protection will be demonstrated by either a reduction or lack of clinical signs normally displayed by an infected host, a quicker recovery time and/or a lowered duration or bacterial titer in the tissues or body fluids or excretions of the infected host.

“Clinical signs” or “clinical symptoms or clinical reactions” for leptospirosis are e.g.: reduced appetite, slow or stiff gait, weakness, vomiting, diarrhoea, reduced skin turgor (indicative of dehydration), pale or yellow mucous membranes (conjunctivae or oral mucosa) and arched back.

“Reduction of the incidence and/or severity of clinical signs” or “reduction in the incidence and/or severity of clinical symptoms” as referred to herein, means reducing the number of infected animals in a group, reducing or eliminating the number of animals exhibiting clinical signs of infection, or reducing the severity of any clinical signs that are present in the animals, in comparison to infection by the wild-type pathogen. For example, such clinical signs include, temperature, general health score, reduced appetite, slow or stiff gait, weakness, vomiting, diarrhoea, reduced skin turgor (indicative of dehydration), pale or yellow mucous membranes (conjunctivae or oral mucosa) and arched back. Suitably, these are reduced in animals receiving the vaccine composition of the present invention by at least 10% in comparison to animals that became infected when not receiving the vaccination

As used herein, “a pharmaceutically acceptable carrier” or “pharmaceutical carrier” includes any and all excipients, solvents, growth media, dispersion media, coatings, adjuvants, stabilizing agents, diluents, preservatives, inactivating agents, antimicrobial, antibacterial and antifungal agents, isotonic agents, adsorption delaying agents, and the like. Such ingredients include those that are safe and appropriate for use in veterinary applications. Suitably, stabilizing agents for use in the present invention include stabilizers for lyophilization or freeze-drying.

“Diluents” may include water, saline, dextrose, ethanol, glycerol, and the like. Isotonic agents may include sodium chloride, dextrose, mannitol, sorbitol, and lactose, among others. Stabilizers may include albumin and alkali salts of ethylenediaminetetraacetic acid, among others.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise.

Suitably the vaccine is administered in a volume of about 0.05 to about 5.0 ml, such as 0.1 to 2.5 ml. Suitably the vaccine is administered in a volume of 0.2 to 2.0 ml, or 0.25 to 1.5 ml, or 0.3 to 1.2 ml, or 0.4 to 1.0 ml or 0.5 to 0.9 ml, or 0.6 to 0.8 ml.

The vaccine may be administered subcutaneously, intramuscular, intraperitoneally, orally, intranasally, intraocularly and/or rectally. Suitably the vaccine is administered subcutaneously, intramuscular, orally, intranasally, intraocularly and/or rectally. Suitably the vaccine is administered subcutaneously, intramuscular, orally, and/or intranasally. Suitably the vaccine is administered subcutaneously, or intramuscular.

Suitably the vaccine is administered in a single dose. Suitably the vaccine is administered in at least 2 doses. The at least 2 doses are administered 2 to 100 days apart, preferably 5 to 60 days apart, more preferably 7 to 50 days apart, more preferably 10 to 40 days apart, more preferably 14 to 30 days apart, and more preferably 15 to 25 days apart. Preferably the at least 2 doses are administered 5 to 40 days apart, preferably 6 to 35 days apart, preferably 7 to 32 days apart, preferably 8 to 30 days apart, preferably 9 to 28 days apart, preferably 10 to 25 days apart, preferably 11 to 22 days apart, preferably 12 to 20 days apart, preferably 13 to 18 days apart, preferably 14 to 16 days apart. Also suitably, at least 2 doses are administered 1 to 12 weeks apart, more suitably 2 to 10 weeks apart, more suitably 2 to 8 weeks apart, more suitably 3 to 6 weeks apart and more suitably 3 to 5 weeks apart.

Preferably the single dose is updated with an annual revaccination. Suitably after the single dose there is a revaccination every 10 to 15 months. Suitably the revaccination is every 11 to 14 months, suitably the revaccination is every 12 to 13 months.

Preferably the at least two doses is updated with an annual revaccination with a single dose. Suitably after the last of the at least two doses there is a revaccination every 10 to 15 months. Suitably the revaccination is every 11 to 14 months, suitably the revaccination is every 12 to 13 months.

Preferably the at least two doses is updated with an annual revaccination with at least 2 doses. Suitably after the last of the at least two doses there is a revaccination every 10 to 15 months. Suitably the revaccination is every 11 to 14 months, suitably the revaccination is every 12 to 13 months.

Suitably, the vaccine comprises an additional antigen that provides immunity against an additional non-leptospiral canine pathogen. Suitably, the additional antigen is selected from the group consisting of canine parvovirus (CPV), canine parainfluenza virus (CPi2), canine distemper virus (CDV), adenovirus, herpesvirus, rabies, canine coronavirus, Bordetella and combinations thereof.

The invention will now be further described by way of the following non-limiting examples.

### EXAMPLES

### General

In total twenty-nine healthy pups with undetectable levels of serum antibodies against serogroup Canicola, Icterohaemorrhagiae, Grippotyphosa and Australis were used. Three groups of seven pups each were vaccinated. Groups 1 and 2 each received a different batch of L4-LV vaccine (one dose is 0.5 ml). Group 3 received a batch of L4 (one dose is 1 ml). The amount of antigen is the same in L4 and L4-LV, the only difference is the volume of the dose. Group 4 was the unvaccinated control group. p1 L4-LV and L4 contain antigens of the following four serovars
Inactivated Leptospira strains

| | | |
|---|---|---|
| serovar Portland-vere (strain Ca-12-000) | 3550-7100 | U* |
| serovar Copenhageni (strain Ic-02-001) | 290-1000 | U* |
| serovar Bratislava (strain As-05-073) | 500-1700 | U* |
| serovar Dadas (strain Gr-01-005) | 650-1300 | U* |

*Antigenic mass ELISA units.

The first vaccination was performed at 6 weeks of age and the second vaccination at 10 weeks of age. The Leptospira challenge was performed 3 weeks after the second vaccination. The dogs were challenged with Leptospira bacteria that were cultured from positive organs of experimentally infected hamsters or homogenate of positive organs.

For evaluation of efficacy after the challenge the following tests or measurements were done: microscopic agglutination test (MAT) to determine serogroup-specific antibody titres against serogroups Australis, Canicola, Grippotyphosa and Icterohaemorrhagiae; body weight;
body temperature;
culturing of the challenge organisms from blood, urine, kidney and liver;
urinalysis;
clinical signs; and
macroscopic and histopathological examination post-mortem.

### Serology

Blood samples for serology and thrombocyte count were taken from all pups five days before the challenge (=16 days after the second vaccination) and at 3, 7, 14, 21 and 28 days post-challenge (pc).

The sera were tested with the microscopic agglutination test (MAT) to determine titres of serogroup-specific agglutinating serum antibodies against serogroups Australis, Canicola, Grippotyphosa and Icterohaemorrhagiae.

Briefly, serial two-fold dilutions of dog serum were incubated with live antigen of each of these serogroups. After this the titre was determined, being the log₂ value of the reciprocal of the highest dilution in which the serum-antigen mixture showed ≥50% agglutinated leptospires. Positive and negative rabbit antisera were used as control sera. The test was considered valid if the titre of the negative control serum was ≤1, and if the titre of the positive control serum was >5 with homologous antigen. All test samples giving agglutination at dilution ≥2 were regarded to be positive.

### Body Temperature

The body temperature was measured three days prior to challenge and daily after challenge until the end of the study.

### Body Weight

The body weight of each dog was determined on the day of arrival (day −12), prior to challenge (days-−5 and twice on day 0) and then daily until the end of the study.

### Reisolation of Challenge Organisms from Blood, Urine, Kidney, and Liver

Blood: Blood samples that were used to obtain culturing results were taken from all dogs 5 days prior to challenge and on days 1, 2, 3, 4, 7, 10, 14 and 21 pc. Aliquots of 0.5 ml directly from the syringe were inoculated into 10 ml of EMJH medium (containing 200 μg/ml 5-fluoro-uracil (5-FU) and 1% (v:v) rabbit serum negative for antibodies against the four relevant serogroups of Leptospira).

For the assessment of the total number of days with positive blood samples per dog, in case of euthanasia and necropsy earlier than the planned necropsy date (day 28 pc) a positive result of the missing blood samples was used. This is needed to avoid an incorrect comparison with the vaccinated groups in which no dog is euthanised and, therefore, no samples are missing.
Urine: Urine samples (at least 2.5 ml) were taken via bladder puncture from all dogs 3 days prior to challenge and on days 3, 7, 14, 21 and 28 after challenge. One ml of urine was directly inoculated into 10 ml of EMJH medium as described above. The remaining 1.5 ml of urine of each dog was used for rapid urinalysis, see below (under Rapid urinalysis). For the assessment of the total number of days with positive urine+kidney samples per dog, in case of euthanasia and necropsy earlier than the planned necropsy date (day 28 post-challenge) a positive result of the missing urine samples was used. This is needed to avoid an incorrect comparison with the vaccinated groups in which no dog was euthanised and, therefore, no samples were missing.
Kidney and liver: After euthanasia, from the cortex of one of the kidneys as well as the liver a piece of 1-2 gram was taken for culture. The fragments—taken aseptically—were placed into 10 ml of culturing medium as described above.

The kidney and liver tissue fragments were homogenized in 10 ml EMJH medium. A 100-fold dilution of each kidney or liver homogenate in EMJH (containing 5-FU and “negative” rabbit serum) was used for culturing.

Cultures of blood, urine, kidney and liver homogenate in EMJH were incubated at 29° C. and observed weekly using dark-field microscopy for the presence of typical Leptospira-shaped, motile bacteria for at least 8 weeks before negative cultures were discarded. The leptospires in some of the positive cultures (at least one per treatment group) were tested for identification of the serogroup by agglutination with the MAT.

### Clinical Signs

On days 1-30 after challenge the dogs were daily checked twice for clinical signs, with special attention for the following non-specific signs associated with canine leptospirosis: reduced appetite, slow or stiff gait, weakness, vomiting, diarrhoea, reduced skin turgor (indicative of dehydration), pale or yellow mucous membranes (conjunctivae or oral mucosa) and arched back.

One or more clinical signs and corresponding clinical score values were assigned to a dog when results of one or more laboratory tests confirmed the presence of leptospirosis. In this way the chance of interference by intercurrent (mild) clinical signs due to causes that are not related to the Leptospira challenge, e.g. intercurrent diarrhoea due to e.g. intestinal opportunistic bacteria or parasites, is significantly reduced.

The following clinical score values were used for the following laboratory-confirmed clinical signs, in order to assess the total clinical score value per dog:

TABLE 1
Matrix of clinical scores

| Clinical sign: | Clinical score: |
|---|---|
| Conjunctival suffusion | 1 |
| Pale conjunctival or oral mucosa | 1 |
| Reduced appetite | 1 |
| Vomiting | 1 |
| Diarrhoea | 1 |
| Jaundice | 1 |
| Reduced skin turgor | 2 |
| No appetite | 2 |
| Weakness | 2 |
| Slow/lethargic or stiff gait | 2 |
| Arched back | 2 |
| Extensive petechiae | 2 |
| Wasting | 2 |
| Euthanasia (earlier than | 150 |
| planned necropsy) | |

The total clinical score of a dog was defined as the sum of all individual scores of each day and all days until the end date of the study for the dogs that were not euthanised earlier than the end date. For the dogs that were euthanised earlier than the planned necropsy date (because the humane endpoint was reached) a total clinical score value of 150 was used.

### Necropsy, Macroscopic Examination and Histopathology

In case of severe clinical signs post-challenge, dogs were euthanised after adequate sedation, immediately followed by post-mortem examination. The same procedure was followed for all surviving dogs on the planned necropsy date. Macroscopic examination was done, particularly with regard to lungs, liver, kidneys, and spleen. Histological examination was done on tissue samples (taken from lesions) from liver, kidneys, spleen and from any organ/tissue with suspected lesions.

Tissue samples from liver, kidneys, spleen, and any organ/tissue with suspected lesions were processed and sections were stained with haematoxylin and eosine (HE) for histopathological examination according to standard procedures. In addition, a Warthin-Starry staining was performed on kidney and liver sections to detect leptospires in kidney and liver tissue. Based on microscopic examination of all slides of all sampled organs and tissues, results were described and interpreted in the context of the specific pathology of canine leptospirosis.

### Example 1 Protection against serovar Grippotyphosa

TABLE 2
Treatment groups

| | | | Age (in weeks) at: | | | |
|---|---|---|---|---|---|---|
| | | Vaccine, | | | Challenge | Challenge¹ |
| Group | | dose | First | Second | (Grippo- | routes and |
| no. | n | volume | vacc. | vacc. | typhosa) | volumes |
| 1 | 5 | L4-LV, | 6 | 10 | 13 | IP: 4.5 ml |
| | | 0.5 ml | | | | IN: 0.4 ml |
| 2 | 6 | L4-LV, | | | | each nostril |
| | | 0.5 ml | | | | Conj.: 0.25 ml |
| 3 | 6 | L4, 1 ml | | | | each eye |
| 4 | 8 | None | NA | NA | | |

¹The challenge material contained 1.4 × 10⁹ bacteriacellsc/ml;
IP, intraperitoneal; IN, intranasal; Conj., instillation into the ventral conjunctival sac of each eye. Each dog received the challenge material in the three ways of administration as indicated. The challenge was done using an in vitro culture of serovar Grippotyphosa strain Duyster.

TABLE 3
Results of blood and urine/kidney culturing from dogs
after challenge with a strain of serovar Grippotyphosa

| | | | | | | | | | | | Total pos. |
|---|---|---|---|---|---|---|---|---|---|---|---|
| | | Blood culture results¹ on days post-challenge (pc): | | | | | | | | | days |
| group | dog | −5 | 1 | 2 | 3 | 4 | 7 | 10 | 14 | 21 | blood cult. |
| 1 | 7149 | − | − | − | − | − | − | − | − | − | 0 |
| | 7203 | − | − | − | − | − | − | − | − | − | 0 |
| | 7297 | − | − | − | − | − | − | − | − | − | 0 |
| | 7335 | − | − | − | − | − | − | − | − | − | 0 |
| | 7441 | − | − | − | − | − | − | − | − | − | 0 |
| | | | | | | | | | | mean | 0.00 S² |
| 2 | 7092 | − | − | − | − | − | − | − | − | − | 0 |
| | 7157 | − | − | + | + | − | − | − | − | − | 2 |
| | 7211 | − | − | − | − | − | − | − | − | − | 0 |
| | 7301 | − | − | − | − | − | − | − | − | − | 0 |
| | 7343 | − | − | − | − | − | − | − | − | − | 0 |
| | 7467 | Euthanized | | | | | | | | | − |
| | | | | | | | | | | mean | 0.40 S² |
| 3 | 7114 | − | − | − | − | − | − | − | − | − | 0 |
| | 7165 | − | − | − | − | − | − | − | − | − | 0 |
| | 7220 | − | − | − | − | − | − | − | − | − | 0 |
| | 7319 | − | − | − | − | − | − | − | − | − | 0 |
| | 7351 | − | − | − | − | − | − | − | − | − | 0 |
| | 7530 | − | + | − | − | − | − | − | − | − | 1 |
| | | | | | | | | | | mean | 0.17 S² |
| 4 | 7122 | − | + | + | + | + | − | − | − | − | 4 |
| | 7181 | − | + | + | + | + | − | − | − | − | 4 |
| | 7254 | − | − | − | + | + | + | − | − | − | 3 |
| | 7327 | − | + | + | + | + | − | − | − | − | 4 |
| | 7360 | − | + | + | + | + | + | − | − | − | 5 |
| | 7424 | − | + | + | + | + | + | − | + | − | 6 |
| | 7491 | − | + | + | + | + | − | − | − | − | 4 |
| | 7548 | − | + | + | + | − | − | − | − | − | 3 |
| | | | | | | | | | | mean | 4.13 |

| | | | | | | | | Kidney cult. | Total pos. |
|---|---|---|---|---|---|---|---|---|---|
| | | Urine culture results¹ on days pc: | | | | | | results¹ on | days urine |
| group | dog | −3 | 3 | 7 | 14 | 21 | 28 | day pc 28 | and kidney³ |
| 1 | 7149 | − | − | − | − | − | − | − | 0 |
| | 7203 | − | − | − | − | − | − | − | 0 |
| | 7297 | − | − | − | − | − | − | − | 0 |
| | 7335 | − | − | − | − | − | − | − | 0 |
| | 7441 | − | − | − | − | − | − | − | 0 |
| | | | | | | | | mean | 0.00 S² |
| 2 | 7092 | − | − | − | − | − | − | − | 0 |
| | 7157 | − | − | − | − | − | − | − | 0 |
| | 7211 | − | − | − | − | − | − | − | 0 |
| | 7301 | − | − | − | − | − | − | − | 0 |
| | 7343 | − | − | − | − | − | − | − | 0 |
| | 7467 | euthanized | | | | | | | − |
| | | | | | | | | mean | 0.00 S² |
| 3 | 7114 | − | − | − | − | − | − | − | 0 |
| | 7165 | − | − | − | − | − | − | − | 0 |
| | 7220 | − | − | − | − | − | − | − | 0 |
| | 7319 | − | − | − | − | − | − | − | 0 |
| | 7351 | − | − | − | − | − | − | − | 0 |
| | 7530 | − | − | − | − | − | − | − | 0 |
| | | | | | | | | mean | 0.00 S² |
| 4 | 7122 | − | − | + | − | − | − | + | 2 |
| | 7181 | − | + | − | + | − | − | + | 3 |
| | 7254 | − | − | + | − | − | + | + | 2 |
| | 7327 | − | − | + | − | − | + | + | 2 |
| | 7360 | − | − | + | + | + | − | + | 4 |
| | 7424 | − | + | + | + | − | − | + | 4 |
| | 7491 | − | + | − | − | + | − | − | 2 |
| | 7548 | − | − | − | − | + | − | − | 1 |
| | | | | | | | | mean | 2.50 |

¹− = negative, + = positive
²S, statistically significant difference between test group and control group (two-sided Wilcoxon test, P < 0.05)
³When urine and kidney cultures are both positive on day 28, then only one positive result is used in the calculation of total pos. Days

TABLE 4
The number of dogs in each group with kidney lesions

| | | | | | |
|---|---|---|---|---|---|
| 1 | 7149 | − | − | − | − |
| | 7203 | − | − | +/− | − |
| | 7297 | − | − | − | − |
| | 7335 | − | − | − | − |
| | 7441 | − | − | − | − |
| 2 | 7092 | − | − | − | − |
| | 7157 | − | − | − | − |
| | 7211 | − | − | − | − |
| | 7301 | − | − | − | − |
| | 7343 | − | − | − | − |
| 3 | 7114 | − | − | − | +/− |
| | 7165 | − | − | − | − |
| | 7220 | − | − | − | − |
| | 7319 | − | − | − | − |
| | 7351 | − | − | − | − |
| | 7530 | − | − | − | − |
| 4** | 7122 | − | − | ++ | − |
| | 7181 | − | − | + | − |
| | 7254 | − | +++ | +++ | − |
| | 7327 | − | ++ | ++ | − |
| | 7360 | − | ++ | ++ | − |
| | 7424 | − | ++ | ++ | +/− |
| | 7491 | − | ++ | ++ | +/− |
| | 7548 | − | − | − | − |

E, intercurrently euthanised
− = no lesions
+/− = minimal/focal lesions
+ = mild focal to multifocal lesions
++ = moderate multifocal lesions
+++ = severe/diffuse lesions
NS = not sampled
**Frequency of moderate/severe kidney lesions (score ++ or more) in control group is significantly higher compared to the vaccinated groups. P-value < 0.05 for each group (Fisher Exact test)

### Example 2 Protection against serovar Australis

TABLE 5
Treatment groups

| | | Vaccine, | Age (in weeks) at: | | | Challenge¹ |
|---|---|---|---|---|---|---|
| Group | | dose | First | Second | Challenge | routes and |
| no. | n | volume | vacc. | vacc. | (Australis) | volumes |
| 1 | 7 | L4-LV, | 6 | 10 | 13 | IP: 4.5 ml |
| | | 0.5 ml | | | | IN: 0.4 ml |
| 2 | 7 | L4-LV, | | | | each nostril |
| | | 0.5 ml | | | | Conj.: 0.25 ml |
| 3 | 7 | L4, 1 ml | | | | each eye |
| 4 | 8 | None | NA | NA | | |

¹The challenge material contained 1.2 × 10⁹ bacteriacells/ml;
IP, intraperitoneal; IN, intranasal; Conj., instillation into the ventral conjunctival sac of each eye. Each dog received the challenge material by the three ways of administration as indicated. The challenge was done using an in vitro culture of serovar Australis strain Dohhilo.

TABLE 6
Total score of laboratory-confirmed clinical signs of Leptospirosis
per dog after challenge with a strain of serovar Australis

| | Total | | | Significance |
|---|---|---|---|---|
| Group | individual | Group | SD | vs control\hat{ } |
| 1 | 0 | 1.0 | 1.7 | p = 0.0439 |
| | 0 | | | |
| | 4 | | | |
| | 0 | | | |
| | 3 | | | |
| | 0 | | | |
| | 0 | | | |
| 2 | 0 | 0.3 | 0.8 | p = 0.0201 |
| | 0 | | | |
| | 0 | | | |
| | 0 | | | |
| | 2 | | | |
| | 0 | | | |
| | 0 | | | |
| 3 | 0 | 1.3 | 2.2 | p = 0.0439 |
| | 4 | | | |
| | 5 | | | |
| | 0 | | | |
| | 0 | | | |
| | 0 | | | |
| | 0 | | | |
| 4 | 0 | 99.6 | 82.3 | — |
| | 0 | | | |
| | 150 | | | |
| | 150 | | | |
| | 150 | | | |
| | 1 | | | |
| | 150 | | | |
| | 150 | | | |

\hat{ }using the two-sided Wilcoxon test

TABLE 7
Results of blood and urine/kidney culturing from dogs
after challenge with a strain of serovar Australis

| | | | | | | | | | | | Total pos. |
|---|---|---|---|---|---|---|---|---|---|---|---|
| | | Blood culture results¹ on days post-challenge (pc): | | | | | | | | | days blood |
| group | dog | −5 | 1 | 2 | 3 | 4 | 7 | 10 | 14 | 21 | cult. |
| 1 | 581 | − | − | − | − | − | − | − | − | − | 0 |
| | 697 | − | + | + | + | − | − | − | − | − | 3 |
| | 751 | − | + | + | + | + | + | − | − | − | 5 |
| | 786 | − | + | + | + | + | + | − | − | − | 5 |
| | 832 | − | + | + | + | − | − | − | − | − | 3 |
| | 891 | − | + | + | + | + | + | − | − | − | 5 |
| | 964 | − | + | + | + | + | + | − | − | − | 5 |
| | | | | | | | | | | mean | 3.71 S² |
| 2 | 492 | − | + | + | + | + | − | − | − | − | 4 |
| | 522 | − | − | + | + | + | − | − | − | − | 3 |
| | 549 | − | + | + | + | + | − | − | − | − | 4 |
| | 573 | − | + | + | + | + | + | − | − | − | 5 |
| | 727 | − | + | + | + | − | − | − | − | − | 3 |
| | 808 | − | + | + | + | + | + | − | − | − | 5 |
| | 859 | − | + | + | + | + | − | − | − | − | 4 |
| | | | | | | | | | | mean | 4.00 S² |
| 3 | 565 | − | + | + | + | + | − | − | − | − | 4 |
| | 701 | − | − | − | − | − | − | − | − | − | 0 |
| | 778 | − | + | + | + | + | − | − | − | − | 4 |
| | 816 | − | + | + | + | + | − | − | − | − | 4 |
| | 867 | − | + | + | + | + | + | − | − | − | 5 |
| | 999 | − | + | + | + | + | + | − | + | − | 6 |
| | 1057 | − | + | + | + | + | − | − | − | − | 4 |
| | | | | | | | | | | mean | 3.86 S² |
| 4 | 484 | − | + | + | + | + | + | − | − | − | 5 |
| | 531 | − | + | + | + | + | − | − | − | − | 4 |
| | 557 | − | + | + | + | + | euthanized⁴ | | | | 8 |
| | 735 | − | + | + | + | + | | | | | 8 |
| | 760 | − | + | + | + | + | | | | | 8 |
| | 824 | − | + | + | + | + | + | + | − | − | 6 |
| | 875 | − | + | + | + | + | euthanized⁴ | | | | 8 |
| | 1006 | − | + | + | + | + | | | | | 8 |
| | | | | | | | | | | mean | 6.88 |

| | | | | | | | | Kidney cult. | Total pos. |
|---|---|---|---|---|---|---|---|---|---|
| | | Urine culture results¹ on days pc: | | | | | | results¹ on | days urine |
| group | dog | −3 | 3 | 7 | 14 | 21 | 28 | day pc 28 | and kidney³ |
| 1 | 581 | − | − | − | − | − | + | − | 1 |
| | 697 | − | − | − | + | + | − | − | 2 |
| | 751 | − | − | + | + | + | + | − | 4 |
| | 786 | − | − | + | + | + | − | − | 3 |
| | 832 | − | − | − | + | + | + | + | 3 |
| | 891 | − | + | + | + | − | − | − | 3 |
| | 964 | − | + | + | − | + | + | + | 4 |
| | | | | | | | | mean | 2.86 S² |
| 2 | 492 | − | − | − | − | − | − | − | 0 |
| | 522 | − | − | − | − | − | − | − | 0 |
| | 549 | − | + | − | + | − | − | − | 2 |
| | 573 | − | + | − | + | − | − | − | 2 |
| | 727 | − | + | − | − | − | − | + | 2 |
| | 808 | − | + | + | + | + | + | − | 5 |
| | 859 | − | − | − | − | + | − | − | 1 |
| | | | | | | | | mean | 1.71 S² |
| 3 | 565 | − | − | − | − | + | + | − | 2 |
| | 701 | − | − | − | − | − | − | − | 0 |
| | 778 | − | − | + | + | + | + | − | 4 |
| | 816 | − | − | + | + | − | + | + | 3 |
| | 867 | − | − | − | + | + | − | + | 3 |
| | 999 | − | − | + | + | + | + | + | 4 |
| | 1057 | − | − | − | − | + | − | − | 1 |
| | | | | | | | | mean | 2.43 S² |
| 4 | 484 | − | − | − | + | + | − | − | 2 |
| | 531 | − | + | + | + | − | + | + | 4 |
| | 557 | − | − | euthanized⁴ | | | | | 4 |
| | 735 | − | + | | | | | | 5 |
| | 760 | − | − | | | | | | 4 |
| | 824 | − | − | + | + | + | + | − | 4 |
| | 875 | − | + | euthanized⁴ | | | | | 5 |
| | 1006 | − | + | | | | | | 5 |
| | | | | | | | | mean | 4.13 |

¹− = negative, + = positive
²S, statistically significant difference between test group and control group (two-sided Wilcoxon test, P < 0.05)
³When urine and kidney cultures are both positive on day 28, then only one positive result is used in the calculation of total pos. Days
⁴Euthanized, although these samples were not available after euthanasia yet the result “+” was counted to avoid an incorrect comparison between groups with and without euthanised dogs due to the incomplete number of blood or urine samples from the euthanised dogs.

TABLE 8
Overview of histopathological lesions related to the
Leptospirosis infection with serovar Australis

| Group | Dog ID | Left kidney | Right kidney | Liver | Lung |
|---|---|---|---|---|---|
| 1 | 581 | − | − | + | NS |
| | 697 | − | − | − | NS |
| | 751 | ++ | ++ | + | NS |
| | 786 | + | + | + | NS |
| | 832 | +/− | − | + | + |
| | 891 | +/− | +/− | + | − |
| | 964 | − | +/− | + | NS |
| 2 | 492 | − | +/− | − | NS |
| | 522 | − | − | − | NS |
| | 549 | − | − | − | NS |
| | 573 | +/− | + | + | NS |
| | 727 | − | +/− | − | NS |
| | 808 | +/− | +/− | +/− | NS |
| | 859 | − | − | +/− | NS |
| 3 | 565 | + | + | + | NS |
| | 701 | − | +/− | + | NS |
| | 778 | + | + | + | NS |
| | 816 | +/− | +/− | +/− | NS |
| | 867 | + | − | + | NS |
| | 999 | ++ | ++ | ++ | NS |
| | 1057 | − | − | +/− | NS |
| 4 | 484 | − | + | − | NS |
| | 531 | +++ | ++ | + | NS |
| | 557 (E) | +++ | +++ | +++ | + |
| | 735 (E) | +++ | +++ | +++ | − |
| | 760 (E) | +++ | +++ | +++ | + |
| | 824 | + | ++ | + | + |
| | 875 (E) | +++ | +++ | +++ | − |
| | 1006 (E) | +++ | +++ | +++ | + |

E, euthanised
− = no lesions
+/− = minimal/focal lesions
+ = mild focal to multifocal lesions
++ = moderate multifocal lesions
+++ = severe/diffuse lesions
NS = not sampled
**Frequency of moderate/severe kidney lesions (score ++ or more) in control group is significantly higher compared to the vaccinated groups. P-value < 0.05 for each group (Fischer's Exact test).

### Example 3 Protection against serovar Icterohaemorrhagiae

TABLE 9
Treatment groups

| | | Vaccine, | Age (in weeks) at: | | | Challenge¹ |
|---|---|---|---|---|---|---|
| Group | | dose | First | Second | Challenge | routes and |
| no. | n | volume | vacc. | vacc. | (Ictero) | volumes |
| 1 | 7 | L4-LV, | 6 | 10 | 13 | IP: 4.5 ml |
| | | 0.5 ml | | | | IN: 0.4 ml |
| 2 | 7 | L4-LV, | | | | each nostril |
| | | 0.5 ml | | | | Conj.: 0.25 ml |
| 3 | 7 | L4, 1 ml | | | | each eye |
| 4 | 8 | None | NA | NA | | |

¹The challenge material contained 6.3 × 10⁸ bacteria cells/ml;
IP, intraperitoneal; IN, intranasal; Conj., instillation into the ventral conjunctival sac of each eye. Each dog received the challenge material in the three ways of administration as indicated. The challenge was done using an in vitro culture of serovar Icterohaemorrhagiae strain Verdun.

TABLE 10
Total score of laboratory-confirmed clinical signs of Leptospirosis
per dog after challenge with a strain of serovar Icterohaemorrhagiae

| Group | Dog ID | Total individual CS* | Group | SD |
|---|---|---|---|---|
| 1 | 5567 | 0 | 0.0 | 0.0 |
| | 5818 | 0 | | |
| | 5958 | 0 | | |
| | 6156 | 0 | | |
| | 6202 | 0 | | |
| | 6334 | 0 | | |
| | 6342 | 0 | | |
| 2 | 5575 | 0 | 0.0 | 0.0 |
| | 5800 | 0 | | |
| | 6148 | 0 | | |
| | 6172 | 0 | | |
| | 6211 | 0 | | |
| | 6318 | 0 | | |
| | 6351 | 0 | | |
| 3 | 5583 | 0 | 0.3 | 0.8 |
| | 5923 | 0 | | |
| | 6121 | 0 | | |
| | 6199 | 2 | | |
| | 6261 | 0 | | |
| | 6296 | 0 | | |
| | 6300 | 0 | | |
| 4 | 5541 | 0 | 56.3 | 77.6 |
| | 5559 | 0 | | |
| | 5931 | 150 | | |
| | 6067 | 0 | | |
| | 6130 | 150 | | |
| | 6181 | 150 | | |
| | 6253 | 0 | | |
| | 6326 | 0 | | |

TABLE 11
Results of blood and urine/kidney culturing from dogs after
challenge with a strain of serovar Icterohaemorrhagiae

| | | | | | | | | | | | Total pos. |
|---|---|---|---|---|---|---|---|---|---|---|---|
| | | Blood culture results¹ on days post-challenge (pc): | | | | | | | | | days blood |
| group | dog | −5 | 1 | 2 | 3 | 4 | 7 | 10 | 14 | 21 | cult. |
| 1 | 5567 | − | − | − | − | − | − | − | − | − | 0 |
| | 5818 | − | − | − | − | − | − | − | − | − | 0 |
| | 5958 | − | − | − | − | − | − | − | − | − | 0 |
| | 6156 | − | − | − | − | − | − | − | − | − | 0 |
| | 6202 | − | + | + | + | + | − | − | − | − | 4 |
| | 6334 | − | − | − | − | − | − | − | − | − | 0 |
| | 6342 | − | + | + | + | + | + | − | − | − | 5 |
| | | | | | | | | | | mean | 1.29 S² |
| 2 | 5575 | − | − | − | − | − | − | − | − | − | 0 |
| | 5800 | − | − | − | − | − | − | − | − | − | 0 |
| | 6148 | − | − | − | − | − | − | − | − | − | 0 |
| | 6172 | − | − | − | − | − | − | − | − | − | 0 |
| | 6211 | − | − | − | − | − | − | − | − | − | 0 |
| | 6318 | − | − | − | − | − | − | − | − | − | 0 |
| | 6351 | − | − | − | − | − | − | − | − | − | 0 |
| | | | | | | | | | | mean | 0.00 S² |
| 3 | 5583 | − | − | − | − | − | − | − | − | − | 0 |
| | 5923 | − | − | − | + | − | − | − | − | − | 1 |
| | 6121 | − | + | + | + | − | − | − | − | − | 3 |
| | 6199 | − | − | − | − | − | − | − | − | − | 0 |
| | 6261 | − | − | − | − | − | − | − | − | − | 0 |
| | 6296 | − | − | − | − | − | − | − | − | − | 0 |
| | 6300 | − | + | + | + | + | − | − | − | − | 4 |
| | | | | | | | | | | mean | 1.14 S² |
| 4 | 5541 | − | + | + | + | + | + | − | − | − | 5 |
| | 5559 | − | + | + | + | + | + | − | − | − | 5 |
| | 5931 | − | + | + | + | + | euthanized⁴ | | | | 8 |
| | 6067 | − | + | + | + | + | + | − | − | − | 5 |
| | 6130 | − | + | + | + | + | euthanized⁴ | | | | 8 |
| | 6181 | − | + | + | + | + | | | | | 8 |
| | 6253 | − | + | + | − | − | − | − | − | − | 2 |
| | 6326 | − | + | + | + | + | + | − | − | − | 5 |
| | | | | | | | | | | mean | 5.75 |

| | | | | | | | | Kidney cult. | Total pos. |
|---|---|---|---|---|---|---|---|---|---|
| | | Urine culture results¹ on days pc: | | | | | | results¹ on | days urine |
| group | dog | -3 | 3 | 7 | 14 | 21 | 28 | day pc 28 | and kidney³ |
| 1 | 5567 | − | − | − | − | − | − | − | 0 |
| | 5818 | − | − | − | − | − | − | − | 0 |
| | 5958 | − | − | − | − | − | − | − | 0 |
| | 6156 | − | − | − | − | − | − | − | 0 |
| | 6202 | − | − | − | − | − | − | − | 0 |
| | 6334 | − | − | − | − | − | − | − | 0 |
| | 6342 | − | + | − | − | − | − | + | 2 |
| | | | | | | | | mean | 0.29 S² |
| 2 | 5575 | − | − | − | − | − | − | − | 0 |
| | 5800 | − | − | − | − | − | − | − | 0 |
| | 6148 | − | − | − | − | − | − | − | 0 |
| | 6172 | − | − | − | − | − | − | − | 0 |
| | 6211 | − | − | − | − | − | − | − | 0 |
| | 6318 | − | − | − | − | − | − | − | 0 |
| | 6351 | − | − | − | − | − | − | − | 0 |
| | | | | | | | | mean | 0.00 S² |
| 3 | 5583 | − | − | − | − | − | − | − | 0 |
| | 5923 | − | − | − | − | − | − | − | 0 |
| | 6121 | − | − | − | − | − | − | − | 0 |
| | 6199 | − | − | − | − | − | − | − | 0 |
| | 6261 | − | − | − | − | − | − | − | 0 |
| | 6296 | − | − | − | − | − | − | − | 0 |
| | 6300 | − | + | − | − | − | − | − | 1 |
| | | | | | | | | mean | 0.14 S² |
| 4 | 5541 | − | − | − | − | − | − | + | 1 |
| | 5559 | − | + | − | − | − | − | − | 1 |
| | 5931 | − | − | euthanized⁴ | | | | | 4 |
| | 6067 | − | − | − | − | − | − | − | 0 |
| | 6130 | − | + | euthanized | | | | | 5 |
| | 6181 | − | + | | | | | | 5 |
| | 6253 | − | + | − | − | − | − | − | 1 |
| | 6326 | − | + | + | − | + | + | + | 4 |
| | | | | | | | | mean | 2.63 |

¹− = negative, + = positive
²S, statistically significant difference between test group and control group (two-sided Wilcoxon test, P < 0.05)
³When urine and kidney cultures are both positive on day 28, then only one positive result is used in the calculation of total pos. Days
⁴Euthanized, although these samples were not available after euthanasia yet the result “+” was counted to avoid an incorrect comparison between groups with and without euthanised dogs due to the incomplete number of blood or urine samples from the euthanised dogs.

TABLE 12
Overview of histopathological lesions related to the Leptospirosis
infection with serovar Icterohaemorrhagiae

| Group | Dog ID | Spleen | Kidney L | Kidney R | Liver | Lung |
|---|---|---|---|---|---|---|
| 1 | 5567 | − | − | − | ++ | NS |
| | 5818 | − | − | − | + | NS |
| | 5958 | − | − | − | + | − |
| | 6156 | − | − | − | + | +/− |
| | 6202 | − | − | − | ++ | − |
| | 6334 | − | − | − | ++ | NS |
| | 6342 | − | − | − | ++ | − |
| 2 | 5575 | − | +/− | +/− | ++ | − |
| | 5800 | − | − | − | + | NS |
| | 6148 | − | − | − | ++ | NS |
| | 6172 | − | − | − | +/− | NS |
| | 6211 | − | − | − | ++ | − |
| | 6318 | − | − | − | + | − |
| | 6351 | − | +/− | +/− | + | NS |
| 3 | 5583 | − | − | − | ++ | NS |
| | 5923 | − | − | − | ++ | NS |
| | 6121 | − | − | − | ++ | NS |
| | 6199 | − | − | − | ++ | +/− |
| | 6261 | +/− | − | − | +/− | − |
| | 6296 | − | − | − | ++ | − |
| | 6300 | − | − | − | ++ | NS |
| 4** | 5541 | − | − | − | + | + |
| | 5559 | − | − | +/− | + | NS |
| | 5931(E) | − | +++ | +++ | +++ | +++ |
| | 6067 | − | − | − | + | NS |
| | 6130(E) | ++ | + | + | +++ | +++ |
| | 6181(E) | +/− | ++ | ++ | +++ | +++ |
| | 6253 | − | − | − | +/− | NS |
| | 6326 | − | + | +/− | ++ | + |

E, intercurrently euthanised
− = no lesions
+/− = minimal/focal lesions
+ = mild focal to multifocal lesions
++ = moderate multifocal lesions
+++ = severe/diffuse lesions
NS = not sampled
**There was no significant difference (Fisher exact test) in the frequency of moderate/severe kidney lesions (score ++ or more) between the control group and the vaccinated groups.

1. A method for inducing an immune response to Leptospira serovar Grippotyphosa in a canine, comprising administering to the canine a vaccine comprising a non-Grippotyphosa Leptospira serovar.

2. The method of claim 1 wherein the vaccine is a multivalent vaccine.

3. The method of claim 1, wherein the vaccine does not comprise Leptospira serovar Grippotyphosa.

4. The method of claim 1, wherein the vaccine comprises Leptospira serovars Portland-vere, Dadas, Copenhageni, and/or Bratislava.

5. The method of claim 1, wherein the method further comprises inducing an immune response in the canine against Leptospira serovars Grippotyphosa, Australis, and Icterohaemorrhagiae.

6. The method of claim 1, wherein 0.1 to 2.5 ml of said vaccine is administered.

7. The method of claim 1, wherein the vaccine is administered subcutaneously.

8. The method of claim 1. wherein the vaccine is administered in at least 2 doses.

9. The method of claim 1, wherein the vaccine is administered in at least 2 doses and the doses are administered at least 5 days apart.

10. The method of claim 1, wherein the vaccine comprises at least one additional antigen that provides immunity against an additional non-Leptospiral canine pathogen.

11. The method of claim 10 wherein the additional antigen is selected from the group of: canine parvovirus (CPV), canine parainfluenza virus (CPi2), canine distemper virus (CDV), adenovirus, herpesvirus, rabies, canine coronavirus and combinations thereof. 12 (New) A method of vaccinating a canine against Leptospira serovar Grippotyphosa, comprising administering to the canine a vaccine composition comprising a Leptospira serovar, where the Leptospira serovar is not Grippotyphosa Leptospira.

13. The method of claim 12, wherein the vaccine is a multivalent vaccine comprising at least two Leptospira serovars, wherein the Leptospira serovars are not Grippotyphosa Leptospira.

14. The method of claim 13, wherein the vaccine comprises Leptospira serovars Portland-vere, Dadas, Copenhageni, and/or Bratislava.

15. The method of claim 14, wherein the method further induces an immune response to any one or a combination of: Leptospira serovar Australis, Grippotyphosa, and Icterohaemorrhagiae.

16. A method to the reduce risk of infection, or to reduce the severity of one or more clinical sign of infection with Leptospira serovar Grippotyphosa in a canine, or both, comprising administering to the canine a composition comprising a vaccine comprising a Leptospira serovar, where the Leptospira serovar is not Grippotyphosa Leptospira.

17. The method of claim 16, wherein the vaccine is a multivalent vaccine comprising at least two Leptospira serovars, wherein the Leptospira serovars are not Grippotyphosa Leptospira.

18. The method of claim 17, wherein the vaccine comprises Leptospira serovars Portland-vere, Dadas, Copenhageni, and/or Bratislava.

19. The method of claim 18, wherein the method further induces an immune response to any one or a combination of: Leptospira serovar Australis, Grippotyphosa, and Icterohaemorrhagiae.
